(19) United States
(12) Patent Application Publication
(10) Pub. No.: US 2014/0038189 A1
(43) Pub. Date: Feb. 6, 2014

(54) METHOD OF DETERMINING HOMOZYGOTE AND HETEROZYGOTE

(71) Applicant: CANON KABUSHIKI KAISHA, Tokyo (JP)

(72) Inventor: Eishi Igata, Utsunomiya-shi (JP)

(73) Assignee: CANON KABUSHIKI KAISHA, Tokyo (JP)

(21) Appl. No.: 13/950,563

(22) Filed: Jul. 25, 2013

(30) Foreign Application Priority Data
Aug. 2, 2012 (JP) 2012-171971

(51) Int. Cl. C12Q 1/68

(52) U.S. Cl. CPC C12Q 1/6844
USPC 435/6.12; 435/287.2

(57) ABSTRACT

In order to accurately determine a homozygote and a heterozygote in a melting curve analysis, provided is an apparatus for detecting a signal from a specimen, the apparatus including a fluidic device including: a fluid path through which the specimen is passable; a reaction unit provided in the fluid path; a heater configured to elevate a temperature of the specimen in the reaction unit so as to perform the melting curve analysis; and a heater driving unit configured to drive the heater. The heater driving unit is configured to drive the heater at a first temperature elevation rate that is equal to or higher than 1° C./s and a second temperature elevation rate that is different from the first temperature elevation rate so that the fluidic device performs multiple times of the melting curve analysis for specimens including the same component.

### BACKGROUND OF THE INVENTION

1. Field of the Invention

The present invention relates to a technology for determining a single nucleotide polymorphism (SNP) by performing a melting curve analysis of a nucleic acid.

2. Description of the Related Art

In the field of analytical chemistry, it is a fundamental matter to acquire desired information such as a concentration and a component, in order to verify a process or results of a chemical reaction or a biochemical reaction, and hence various devices and sensors have been invented to acquire such pieces of information. A concept referred to as “micro total analysis systems (μ-TAS)” or “lab on a chip” has been known, which reduces sizes of such devices and sensors to a microscale level to achieve all processes up to acquisition of the desired information on a microdevice. This is a concept aiming at undergoing a process such as specimen purification or a chemical reaction by causing a collected raw material or a crude specimen to pass through a fluid path in the microdevice and finally acquiring the concentration of a component or the like included in a chemically synthesized product or the specimen. Such a microdevice that is responsible for the analysis and the reaction inevitably handles a trace of solution or gas, and hence it is often referred to as “micro-fluid-path device” or “microfluidic device”.

Comparing to a desktop-sized analysis device of a conventional technology, the use of the micro-fluid-path device leads to reduction in volume of the fluid in the device, and hence reduction in required amount of a reagent and a reduction in reaction time due to a reduction in amount of an analyte to be analyzed to a microscale level are expected. With an acknowledgement of the advantage of the micro-fluid-path device, technologies involved in the μ-TAS have been attracting attention.

As one of the technologies involved in the μ-TAS, research and development on an application thereof to a technology that handles a nucleic acid are actively proceeding. By identifying that an activity of a protein, of which architecture a gene sequence determines via an amino acid, is a factor for a genetic disease or a factor for determining a drug metabolic capacity, it is expected that a diagnosis of the genetic disease or the drug metabolic capacity can be achieved from the gene sequence. For example, with respect to multiple individuals each having different drug metabolic capacity, the difference in the capacity may be generated by a difference in only a single nucleotide. The difference in the single nucleotide, which is referred to as “single nucleotide polymorphism”, has become one of the key words for the future personalized medicine. In such a test, by using the micro-fluid-path device, results can be rapidly derived with a small amount of specimen.

In a conventional SNP detection, a Taqman probe is used to determine whether a gene of interest is a wild type, a homozygous mutant type, or a heterozygous mutant type from a wavelength and an intensity of florescence. In contrast to this, there is a thermal analysis method that involves making a determination based on a melting curve of the nucleic acid without using the expensive Taqman probe. The thermal analysis method is excellent in convenience because measurement can be carried out only with an intercalator florescent dye of a single color. Further, results can be rapidly output by performing the melting curve analysis in a micro fluid path (see Japanese Patent Translation Publication No. 2009-525759).

A degree of difficulty in determining a genotype from the melting curve analysis mainly depends on an accuracy of a differential curve obtained from the melting curve. The differential curve is influenced by an elevation/descent rate of a temperature, and with respect to different specimens, it is not always desired to set the same temperature elevation rate. In determining the genotype, it is preferred to rapidly elevate the temperature in some cases, and it is preferred to slowly elevate the temperature in other cases.

### SUMMARY OF THE INVENTION

The present invention provides a device for efficiently determining a homozygote and a heterozygote from a peak of a differential value by setting an elevation/descent rate suitable for a specimen by using a fluid path.

According to one embodiment of the present invention, there is provided a fluidic device, including: a fluid path through which a specimen is passable; at least one reaction unit provided in the fluid path; a heater configured to elevate a temperature of the specimen in the at least one reaction unit; and a heater driving unit. The heater driving unit is configured to drive the heater at a first temperature elevation rate that is equal to or higher than 1° C./s and a second temperature elevation rate that is different from the first temperature elevation rate so that the fluidic device performs multiple times of a melting curve analysis for specimens including the same component.

According to one embodiment of the present invention, there is provided a method of detecting a signal from a specimen, the method including: elevating temperatures of specimens including the same component in a fluidic device at a first temperature elevation rate that is equal to or higher than 1° C./s and a second temperature elevation rate that is different from the first temperature elevation rate; and detecting the signal generated from each of the specimens during the elevation of the temperatures of the specimens.

According to the present invention, the peak of the differential value obtained from the melting curve can be easily fixed. With this configuration, the differential curves of the homozygote and the heterozygote can be easily determined.

Further features of the present invention will become apparent from the following description of exemplary embodiments with reference to the attached drawings.

### BRIEF DESCRIPTION OF THE DRAWINGS

FIG. 1 is a schematic diagram illustrating a measurement principle of a fluidic device according to an embodiment of the present invention.

FIGS. 2A, 2B, 2C, and 2D are schematic diagrams illustrating a principle of a differential curve according to embodiments of the present invention.

FIG. 3 is a schematic diagram illustrating a measurement principle of a fluidic device according to another embodiment of the present invention.

### DESCRIPTION OF THE EMBODIMENTS

Exemplary embodiments of the present invention are described in detail below.

A fluidic device according to the embodiments of the present invention includes a fluid path through which a specimen is passable, a reaction unit provided in the fluid path, a heater configured to elevate a temperature of the specimen in the reaction unit in order to perform a melting curve analysis, and a heater driving unit configured to drive the heater. The heater driving unit is configured to drive the heater at a first temperature elevation rate that is equal to or higher than 1° C./s and a second temperature elevation rate that is different from the first temperature elevation rate so that the fluidic device performs multiple times of the melting curve analysis for specimens including the same component.

A detection method according to the embodiments of the present invention, which is a method of detecting a signal from the specimen, includes elevating the temperatures of the specimens including the same component in the fluidic device at the first temperature elevation rate that is equal to or higher than 1° C./s and the second temperature elevation rate that is different from the first temperature elevation rate, and detecting the signal generated from each of the specimens during the elevation of the temperatures of the specimens.

It is preferred that the fluidic device include a branch in the fluid path through which the specimen is passable, elevate the temperatures of the specimens including the same component at different positions in the device by the driving of the heater, and detect the signal generated from each of the specimens during the elevation of the temperatures.

The present invention provides a fluidic device for easily identifying a genotype, and hence details of the genotype are described first. Under a room temperature, a gene forms a double strand, and the double strand forms a complementary pair. This state is referred to as a “homozygote”. On the other hand, a part of the complementary strands may be a pair that cannot make a hydrogen bond of a Watson-Crick type base pair. This state is referred to as a “heterozygote”. For example, as an example of the heterozygote, when there is a base other than thymine or uracil at a complementary position of an adenine base, the hydrogen bond cannot be formed so that no bond is generated.

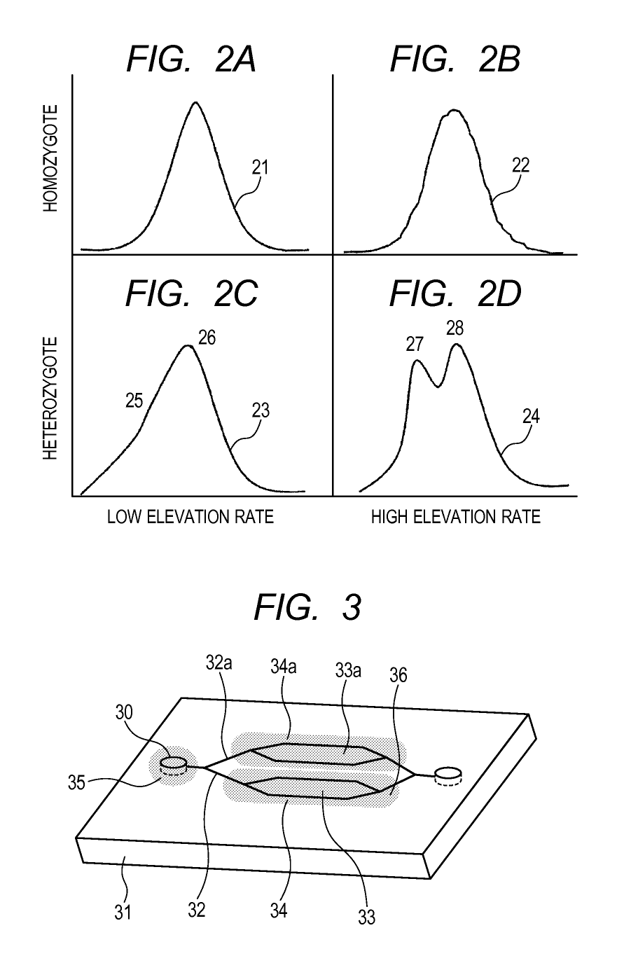

In FIGS. 2A, 2B, 2C, and 2D, the horizontal axis represents the temperature, and the vertical axis represents a negative value of the differential value (hereinafter referred to as a “differential curve”). Those figures are classified by a homozygote, a heterozygote, and a level of the temperature elevation rate at the time of the melting curve analysis. The temperature of the horizontal axis increases from the left to the right.

In the embodiments of the present invention, the temperature elevation rate that is equal to or higher than 1° C./s is referred to as a “high temperature elevation rate”, and the temperature elevation rate that is lower than 1° C./s is referred to as a “low temperature elevation rate”.

Firstly, regarding a nucleic acid of a homozygote, when the melting curve analysis is performed at the low temperature elevation rate, the melting curve is measured and the negative value of the differential value is obtained, a differential curve 21 having one peak as illustrated in FIG. 2A is obtained. This is because the number of measurement points that can be measured is increased for each temperature when the temperature elevation rate is low, and hence the differential curve is likely to be a smooth curve by an appropriate filtering due to an effect of an averaging. In contrast to this, FIG. 2B illustrates a differential curve 22 when the melting curve analysis is performed on the sample of the homozygote at the high temperature elevation rate. Since the temperature elevation rate is high, the number of the measurement points is decreased for each temperature, and hence it is likely to be influenced by a noise. The peak of the differential value indicates a melting temperature Tm of the nucleic acid.

On the other hand, regarding a heterozygote, when the melting curve analysis is performed at the high temperature elevation rate as illustrated in FIG. 2D, multiple peaks are generated in a differential curve 24. However, when the temperature elevation rate is low, only one high peak 26 appears as illustrated in FIG. 2C, and a slight shoulder 25 may appear on a differential curve 23. Normally, the nucleic acid is amplified before performing the melting curve analysis. During the amplification, in the case of the heterozygote, a double strand of the homozygote and a double strand of the heterozygote are formed. The double strand of the heterozygote is melted at a lower temperature due to the lack of the hydrogen bond, which appears as a peak 27 in FIG. 2D. When the temperature is further elevated, the melting of the double strand of the homozygote becomes prominent, which forms a different peak 28 in FIG. 2D. The multiple peaks are a characteristic of the differential curve of the heterozygote. However, in the state illustrated in FIG. 2C, the differential curve is similar to that of the homozygote having one peak, although the slight shoulder 25 can be observed. This indicates that a heterozygote can be mistakenly determined to be a homozygote in a test result. Therefore, the low temperature elevation rate is suitable for determining the homozygote, and the high temperature elevation rate is suitable for determining the heterozygote.

The reason for the generation of the state illustrated in FIG. 2C resides in a mechanism in which the nucleic acid of the double strand becomes a single strand by being melted mainly at a temperature near Tm. Although the shoulder 25 is a signal from the heterozygote portion in the product of the amplification of the heterozygote, when the temperature elevation rate is low, a period of time for which the temperature stays near Tm is increased. Although the heterozygote portion becomes the single strand at the temperature of the shoulder 25, when the temperature is further elevated to a temperature near the peak 26, the melted heterozygote portion is recombined during the elevation of the temperature to form a homozygote, and hence the melting of the newly formed homozygote is simultaneously proceeding. That is, the base of the heterozygote portion that is once melted contributes to the signal again. Therefore, the value of the peak 26 is further increased, and the shoulder 25 is relatively small, possibly resulting in a curve that is similar to the differential curve of the homozygote.

The mechanism in which the homozygote and the heterozygote are hard to determine is described so far. However, in a practical specimen test, it is not identified in advance whether a composition of a sample is a homozygote or a heterozygote, and hence these two genotypes need to be reliably distinguished from each other. The fluidic device for performing this determination is further described below.

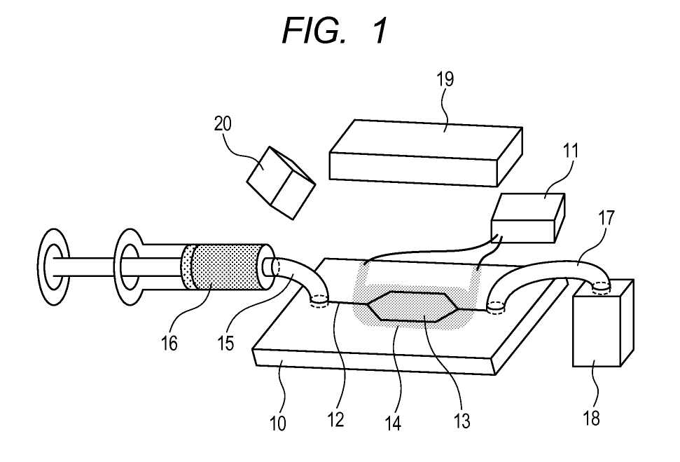

As illustrated in FIG. 1, a fluidic device 10 according to an embodiment of the present invention includes a heater driving unit 11, a fluid path 12, a reaction unit 13, and a heater 14. Further, a tube 15 for supplying a specimen solution is connected to the fluidic device 10, and the solution is supplied by a syringe 16. The solution may also be supplied by using an automatic syringe drive. The solution after a reaction is guided to a waste fluid container 18 through a waste fluid tube 17. An imaging apparatus or a photo-detector 19 is provided above or below the reaction unit 13, which records a reaction in response to light radiated from an irradiation device 20. It is preferred that the heater 14 be provided inside the device so as to shorten a response time of a following temperature of the reaction unit 13. In order to build the heater 14 in the fluidic device, an electrically conductive thin film of metal or ITO is first deposited in proximity to the reaction unit 13 by evaporation or sputtering. An insulation material such as silicon dioxide is then formed, since, when the heater thin film is brought into direct contact with the fluid, a bubble may be generated at the time of elevation of the temperature. Lastly, the heater 14 is built in the fluidic device 10 by bonding the reaction unit with a substrate having a shape of the fluid path.

It is preferred that a material for the fluidic device 10 include a material having a high thermal conductivity, such as silicon (thermal conductivity: 168 W/mK), in order to rapidly perform elevation of the temperature and cooling. The material for the fluidic device 10 may be glass (thermal conductivity: 1 W/mK), considering a purpose and a throughput. However, if a plastic material is used, the thermal conductivity often becomes lower than 1 W/mK, which is not suitable for the rapid elevation of the temperature and cooling.

In FIG. 1, a nucleic acid specimen which is unknown whether it is a homozygote or a heterozygote is supplied to the reaction unit 13 by the syringe 16 through the tube 15 and the fluid path 12. The heater 14 is driven to perform a thermal analysis at a predetermined temperature elevation rate from a start temperature of the melting curve analysis, and a florescence from the nucleic acid specimen at this time is recorded by the photo-detector 19. The temperature is elevated up to about 90° C. by the heater 14 to denature the nucleic acid, then a cooling is performed down to the start temperature of the melting curve analysis again, and the melting curve analysis is performed again at a different temperature elevation rate. In this manner, by performing two times of the melting curve analysis on the same sample at different temperature elevation rates, even when the composition of the nucleic acid specimen is unknown, the melting curve analysis can be performed with respect to the specimen at an appropriate temperature elevation rate.

In a conventional desktop-sized device that is configured to perform the melting curve analysis, the cooling takes a long period of time so that the measurement time needs to be prolonged significantly in order to obtain the melting curve multiple times. Further, regarding the temperature elevation rate, in order to uniformly elevate a temperature of a well plate, the melting curve can only be obtained at the temperature elevation rate as low as, for example, 0.01° C./s. In addition, if a system includes an external heat source, it takes a long period of time to set the temperatures of the well plate and the inside of the fluid path equal to the temperature of the heat source even when the temperature of the heat source is controlled, which is another factor that urges the temperature elevation rate to be set to a low value.

On the other hand, when the heater 14 is accommodated inside the fluidic device 10, the heater 14 can be arranged in proximity to the reaction unit 13, and hence a difference in temperature between the heater 14 and the reaction unit 13 is decreased, which facilitates control. Further, a temperature change of the heat source can be rapidly transferred to the reaction unit. Therefore, the rapid elevation of the temperature and cooling can be performed, and multiple times of the melting curve analysis can be executed without taking a considerable length of time.

That is, by using the fluidic device, the melting curve analysis can be performed with a setting suitable for distinguishing the heterozygote and the homozygote from each other in a rapid manner. Further, in order to increase the accuracy of the melting curve analysis, the fluidic device according to the embodiments of the present invention can also be used to perform multiple times of the melting curve analysis in a temperature range suitable for detecting the heterozygote or a temperature range suitable for detecting the homozygote.

It is preferred that the fluidic device be configured to flow a fluid of microliter (μL) size or nanoliter (nL) size through the fluid path, and that the fluidic device be a so-called microfluidic device. Further, a size of the fluid path is not particularly limited, but the fluid path may have a width of 5 μm to 500 μm, a height of 5 μm to 500 μm, and a length of 1 mm to 100 mm.

The present invention is described in more detail below with reference to examples.

### EXAMPLES

The present invention is described in more detail below with reference to examples. However, the following examples only describe the present invention in more detail, and the embodiments of the present invention are not limited to the following examples.

### Example 1

Example 1 relates to a method of determining the homozygote and the heterozygote by performing two times of the melting curve analysis on the same nucleic acid specimen at different temperature elevation rates. In this method, the high temperature elevation rate is applied first.

The high temperature elevation rate has an advantage in detecting the heterozygote. Therefore, when the heterozygote can be determined at the high temperature elevation rate, it is not necessary to subsequently perform the melting curve analysis at the low temperature elevation rate, and hence the measurement time can be shortened.

The heterozygote is generated with a heterozygous mutant type of the gene. However, in a genetic disease that is caused by recessive inheritance even when the gene is mutated, a patient may lead his or her everyday life without any inconvenience. However, when a baby is born between a man and a woman both having the heterozygous mutant type, the baby has a possibility of an expression of the recessive inheritance developing the genetic disease with ¼ of probability. Therefore, a genetic disease such as cystic fibrosis can be arbitrarily tested before a baby is born by performing a carrier screening.

By using the fluidic device according to the embodiments of the present invention, a diagnosis can be performed in a rapid manner to determine whether a patient is a carrier of a genetic disease. In a diagnosis of the genetic disease carrier, since there are many diseases that, if a patient is a homozygous mutant type, it causes difficulty for the patient to live as long as a child-bearing age when the diseases are developed, in many cases the homozygous mutant type is not determined at the test, and only the heterozygous mutant type is determined. Hence it is desired to first perform the melting curve analysis at the high temperature elevation rate. Thereafter, when the determination does not indicate the heterozygote, the temperature elevation rate is set to a lower value for a confirmation to determine whether or not the genotype is a homozygote based on the number of peaks of the differential curve.

In order to execute the melting curve analysis of a patient-derived nucleic acid specimen, the gene needs to be amplified as a pre-process before the melting curve analysis. The fluidic device 10 illustrated in FIG. 1 may perform a process required for the amplification at the reaction unit 13. For example, a thermal cycle is applied by the heater 14 to perform a polymerase chain reaction, and the melting curve analysis can be executed at the same reaction unit as it is. If the gene amplification can also be performed at the same reaction unit, the amplified nucleic acid not exposed to the outside, and hence a contamination in the measurement environment can be reduced and an error can be reduced because the specimen can be handled without intervention of a person.

In a desktop-sized device, the temperature elevation rate is about 0.4° C./s even at the highest. However, the heater arranged inside the fluidic device can set the temperature elevation rate to about 1° C./s to 10° C./s. As the temperature elevation rate is higher, the peak at the heterozygous mutant type portion of the heterozygote, i.e., the peak 27 in FIG. 2D is increased, and hence the heater built in the fluidic device is suitable for detecting the heterozygote.

### Example 2

Example 2 relates to an example of performing the melting curve analysis at the low temperature elevation rate first and subsequently performing the melting curve analysis at the high temperature elevation rate.

A gene having a homozygous mutant in the single nucleotide polymorphism is caused by the recessive inheritance, which is a factor of a specific genetic disease. Some of these genes cause a serious disorder such as congenital metabolic abnormality or multiple organ failure. In particular, a check of the congenital metabolic abnormality is one of the major tests for a newborn baby.

When a symptom of a disease, which is caused by the recessive inheritance, is suspected, there is a possibility that the gene is a homozygous mutant type. At this time, in order to detect the homozygous mutant type, it suffices to obtain the melting curve at the low temperature elevation rate first. The differential curve 21 having one peak as illustrated in FIG. 2A is obtained with this measurement, but a possibility of being the heterozygote as illustrated in FIG. 2C cannot be excluded at this time. Therefore, in order to exclude the possibility of being the heterozygote, it is preferred to perform the second melting curve analysis at the high temperature elevation rate. If the number of peaks is one as illustrated in FIG. 2B at the second melting curve analysis, it can be said that the patient has the gene of the homozygous mutant type. Further, if the temperature accuracy of the measurement device is equal to or lower than 0.1° C., it is also possible to determine the wild type and the homozygous mutant type.

When the melting curve analysis is performed at the low temperature elevation rate first, there is a further advantage. In the process of amplifying the nucleic acid, for example, when the polymerase chain reaction is used, elongation of DNA is often performed for a relatively long period of time at an activation temperature (65° C. to 72° C.) of a DNA polymerase, at the last step of the amplification. In this state, the DNA of the heterozygote is formed of base pairs of two sets of the homozygote. If the melting curve analysis is performed in a range from 60° C. to 90° C. after the above-mentioned process, the differential curve is likely to generate one peak.

On the other hand, when the temperature is lowered to 60° C. after once denaturing all the DNAs in the solution to a single strand at a temperature of about 95° C. after the amplification process, a base pair of the homozygote and the heterozygote is formed in the solution. When the melting curve analysis is performed in this state, the differential curve includes two peaks, and the heterozygote can be easily determined.

Therefore, when the melting curve analysis is first performed at the low temperature elevation rate, the heterozygote can be easily confirmed at the second melting curve analysis, because the nucleic acid is once denatured.

Further, a cooling rate of the solution after the DNA is denatured is related to a formation of the heterozygote. As the cooling rate is higher, the formation of the heterozygote is expedited. The fluidic device can achieve this situation without arranging an external cooling mechanism. The solution in the fluidic device can rapidly follow its environmental temperature. Therefore, by setting the temperature of the heater in proximity to the fluid path to, for example, 60° C., the solution can be cooled in 1 to 2 seconds from 95° C. at which the DNA is denatured.

### Example 3

The present invention is not limited to a scheme in which multiple times of the melting curve analysis are performed at the same position for the same specimen, but if the melting curve analysis can be performed for the same specimen at different temperature elevation rates, multiple melting curve analyses can be performed independently at two or more different positions in the fluidic device. This enables the multiple melting curve analyses to be performed at the same time, which shortens the processing time.

In FIG. 3, a fluidic device 31 is designed to supply the specimen solution to reaction units 33 and 33a arranged in parallel to each other through a fluid path 32 or a branched fluid path 32a. Heaters 34 and 34a are arranged in proximities to the reaction units 33 and 33a, respectively. The fluid paths 32 and 32a can branch the solution of the same specimen, and hence the same specimen injected from a specimen solution inlet 30 can be guided to the two reaction units. A heater 35 arranged in proximity to the inlet can be controlled independently of the heaters 34 and 34a. In order to reduce a temperature interference between the heaters 34 and 34a, a space between the reaction units 33 and 33a may be cut out.

The specimen solution is supplied to the inlet 30. When the specimen solution includes the nucleic acid of a sufficient concentration, there is no need for amplifying the nucleic acid by driving the heater 35. However, when the concentration of the nucleic acid in the specimen solution is low, the heater 35 is driven with respect to a solution including the specimen and a reagent required for the amplification so as to amplify the nucleic acid. The solution including the nucleic acid of the sufficient concentration is branched at a branch point, and then supplied to the reaction units 33 and 33a through the fluid paths 32 and 32a, respectively. The series of movement of the solution can be achieved by a pressure adjustment of a pump connected to the inlet or an outlet. When the amplification is performed at the inlet 30, a valve may be provided between the inlet 30 and the branch point of the fluid paths 32 and 32a to prevent the reagent from moving to the reaction units.

At the reaction unit 33, if the melting curve analysis is performed, for example, at the temperature elevation rate of 0.05° C./s for the heater 34 and at the temperature elevation rate of 1.0° C./s for the heater 34a, a throughput is improved because the processes can be performed in parallel. A reaction at the reaction unit 33a for testing the heterozygote is completed in 30 seconds in a temperature change range from 60° C. to 90° C. Thereafter, the analysis at the reaction unit 33 is stopped, and the specimen solution is discharged to an outlet 36 so that a measurement of the next specimen can be started. Before moving the solution to the fluid paths 32 and 32a, a product of the amplification may be once denatured at the inlet 30.

A reaction at the reaction unit 33 for testing the homozygote takes 600 seconds in the temperature change range from 60° C. to 90° C. However, the confirmation that the specimen is not the heterozygote is obtained from the result of the reaction unit 33a before 600 seconds elapse, and hence it is possible to reduce the possibility of misjudging the homozygote and the heterozygote when the number of peaks of the differential curve is one.

That is, in the conventional device, it takes 600 seconds to output the result under the above-mentioned condition, and in addition there is a possibility of misjudging the result. In contrast to this, if the melting curve analysis is performed at different temperature elevation rates in parallel in the fluidic device, the results are obtained in 30 seconds in the case of the heterozygote and in 600 seconds in the case of the homozygote. However, in both cases, the misjudgment of the results is considerably reduced.

In addition, the specimen solution is branched conveniently without intervention of a human hand, which is also an advantage of using the fluidic device instead of the solution inside the well plate.

The present invention can be applied to an apparatus for performing a chemical reaction and a chemical analysis of a nucleic acid.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-171971, filed Aug. 2, 2012, which is hereby incorporated by reference herein in its entirety.

What is claimed is:

1. A fluidic device, comprising:
a fluid path through which a specimen is passable;
at least one reaction unit provided in the fluid path;
a heater configured to elevate a temperature of the specimen in the reaction unit; and
a heater driving unit,
wherein the heater driving unit is configured to drive the heater at a first temperature elevation rate that is equal to or higher than 1° C./s and a second temperature elevation rate that is different from the first temperature elevation rate so that the fluidic device performs multiple times of a melting curve analysis for specimens including the same component.

2. The fluidic device according to claim 1, wherein the specimen comprises a nucleic acid.

3. The fluidic device according to claim 2, wherein the fluidic device is configured to detect a heterozygote through driving of the heater at the first temperature elevation rate.

4. The fluidic device according to claim 2, wherein:
the second temperature elevation rate is lower than 1° C./s; and
the fluidic device is configured to detect a homozygote through driving of the heater at the second temperature elevation rate.

5. The fluidic device according to claim 2, further comprising another heater configured to amplify the nucleic acid before performing the melting curve analysis.

6. The fluidic device according to claim 2, further comprising a heater configured to denature the nucleic acid before performing the melting curve analysis.

7. The fluidic device according to claim 1, wherein the heater driving unit is configured to drive the heater at the first temperature elevation rate, and then drive the heater at the second temperature elevation rate.

8. The fluidic device according to claim 1, wherein the heater driving unit is configured to drive the heater at the second temperature elevation rate, and then drive the heater at the first temperature elevation rate.

9. The fluidic device according to claim 1, wherein the at least one reaction unit provided in the fluid path comprises only one reaction unit.

10. The fluidic device according to claim 1, wherein:
the at least one reaction unit provided in the fluid path comprises at least two reaction units arranged in parallel to each other;
the heater of the fluidic device comprises at least two heaters; and
the at least two heaters are configured to elevate temperatures of the specimens including the same component at one of the first temperature elevation rate and the second temperature elevation rate in the at least two reaction units, respectively in an independent manner.

11. The fluidic device according to claim 1, wherein the fluid path has a width of 5 μm to 500 μm, a height of 5 μm to 500 μm, and a length of 1 mm to 100 mm.

12. An apparatus for detecting a signal from a specimen, the apparatus comprising:
the fluidic device according to claim 1; and
a signal detection unit configured to detect the signal generated from the specimen during the elevation of the temperature of the specimen through driving of the heater.

13. A method of detecting a signal from a specimen, the method comprising:
elevating temperatures of specimens including the same component in a fluidic device at a first temperature elevation rate that is equal to or higher than 1° C./s and a second temperature elevation rate that is different from the first temperature elevation rate; and
detecting the signal generated from each of the specimens during the elevation of the temperatures of the specimens.

14. The method according to claim 13, wherein:
the elevating comprises elevating the temperatures of the specimens including the same component at one of the first temperature elevation rate and the second temperature elevation rate in at least two reaction units arranged in a fluid path in parallel to each other, respectively in an independent manner; and
the detecting comprises detecting the signal generated from the each of the specimens in the at least two reaction units during the elevation of the temperatures of the specimens, respectively.
